(19) United States
(12) Patent Application Publication
(10) Pub. No.: US 2010/0203022 A1
(43) Pub. Date: Aug. 12, 2010

(54) METHODS AND COMPOSITIONS FOR DELIVERING THERAPEUTIC AGENTS TO THE CENTRAL NERVOUS SYSTEM

(76) Inventor: Scott P. Falci, Morrison, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 12/702,873

(22) Filed: Feb. 9, 2010

Related U.S. Application Data
(60) Provisional application No. 61/151,116, filed on Feb. 9, 2009.

(51) Int. Cl. A61K 35/12; A61P 25/00

(52) U.S. Cl. 424/93.7

(57) ABSTRACT

Methods are provided for targeting therapeutic agents to an injury site within the central nervous system.

### CROSS-REFERENCE TO RELATED APPLICATIONS

This application claims benefit under 35 U.S.C. §119(e) to U.S. Provisional Application No. 61/151,116, filed Feb. 9, 2009, which is incorporated herein by reference in its entirety.

This application is related to U.S. patent application Ser. No. 10/232,908 titled “METHOD FOR ERADICATING PAIN OF CENTRAL ORIGIN RESULTING FROM SPINAL CORD INJURY,” filed Aug. 30, 2002, which is hereby incorporated by reference herein in its entirety.

### BACKGROUND OF THE INVENTION

a. Field of the Invention

The invention generally relates to methods and compositions for therapy in the central nervous system (CNS) and more particularly to methods for identifying target sites for delivery of these therapies into the CNS of a patient in need thereof.

b. Background Art

Acute and/or chronic injury to the spinal cord often debilitates patients, altering the patient's everyday life. Loss of motion due to nerve damage and/or disabling pain often results from spinal cord injury. Recovered function from damaged or necrotic tissue within the spinal cord has proven to be problematic, often leaving patients with spinal injuries in a state of chronic disability for the remainder of their lives.

Over the past several years a number of studies have focused on restorative and regenerative therapies with regard to spinal cord injury. These studies have focused on the potential for restoring or regenerating damaged tissue using animal models (See Schwab, Repairing the Injured Spinal Cord, SCIENCE 295 (5557):1029-1031; Almudena et al., Functional Recovery of Paraplegic Rats and Motor Axon Regeneration in Their Spinal Cords by Olfactory Ensheathing Glia, NEURON 25(2), (2000), 425-435; Kwon et al., Animal Models Used in Spinal Cord Regeneration Research, SPINE, 27(14) (2002):1504-1510). However, translation of restorative and regenerative therapies in spinal cord injury, derived from animal research, to the human condition, remains a significant challenge.

A significant difficulty faced in providing adequate restorative and regenerative therapies to the CNS is directed at how to target therapies within an acutely or chronically injured site to maximize the effect of the therapy, i.e., one must have a method of targeting, during surgery on, or manipulation of, the CNS, for placement of therapeutic agents within the injury site. For example, after spinal cord injury, various degrees of cell death, partial injury, progressive injury, demyelization, glial scarring, and hematoma formation may occur. (See, Åkesson, Human Spinal Cord Transplantation, Experimental and Clinical Application, Stockholm 2000, Division of Geriatric Medicine, NEUROTEC, pp 7-16). Each of these different cellular environments provides different challenges for useful therapeutic agents, including restorative cellular therapies. For example, for a therapeutic agent to be effective, the cellular content and environment must be optimized for maximal potential benefit of that therapy. In this regard, certain therapies, e.g., drugs or stem cell compositions, would have little benefit if they would not have the capacity or numbers to influence the injury, or be delivered into a spinal region with a hostile environment for cell survival, e.g., a hematoma. In such circumstances the cells will often undergo apoptosis or become quiescent.

Against this backdrop the present disclosure was developed.

### SUMMARY

In various embodiments, methods of delivering a therapeutic agent to specific locations in the central nervous system (CNS) of a patient are provided. The methods combine imaging the site of damage or injury, detecting neuroelectrical activity at the injury, and then delivering therapeutic agents to the sites. In certain embodiments, a preoperative image of the CNS or a portion thereof (e.g. the spinal cord) of a patient is acquired to determine the boundaries of a region of damaged tissue. One or more intraoperative ultrasound images of an injury region are then obtained. The neuroelectrical activity at and surrounding the injury site is measured. In certain cases, zones of active neuron activity, inactivity, hypoactivity and/or hyperactivity are detected. The ultrasound images are correlated with the measured neuroelectrical activity at and surrounding the injury site to identify one or more delivery zones for said therapeutic agent.

Various therapeutic agents can promote axonal regeneration or remyelination, inhibit nerve impulses from dissipating in demyelinated areas, or inhibit exotoxic shock. In still further variations the therapeutic agents are stem cells.

### BRIEF DESCRIPTION OF THE DRAWINGS

FIG. 1 depicts an exemplary MRI from a patient having a spinal cord injury.

FIG. 2 depicts an exemplary intraoperative ultrasound from a patient having a spinal cord injury.

FIG. 3A depicts an electrophysiologic recording of an injured spinal cord at a contusion site analysis of the dorsal grey matter (DREZ) within an injury site.

FIG. 3B depicts an electrophysiologic recording of spontaneous neuroelectrical activity for normal or healthy cells in the dorsal grey matter of the spinal cord caudal to an injury site

FIG. 3C depicts a region of cells in the grey matter (DREZ) adjacent to, or bordering, the acute injury showing hyperactive neuroelectrical activity.

FIG. 4A depicts an electrophysiologic recording of uninjured tissue in the spinal thalamic tract of an individual.

FIG. 4B depicts an electrophysiologic recording of uninjured tissue in the lateral posterior column of an individual.

FIG. 4C depicts an electrophysiologic recording of normal, uninjured tissue in the medial posterior column.

FIG. 5A depicts an electrophysiologic recording of the corticospinal tract of a patient.

FIG. 5B depicts an electrophysiologic recording of the dorsal grey matter of a patient.

FIG. 6 depicts an electrical recording of extreme myelomalacia and cystic necrosis in the tissue of a patient.

FIG. 7 depicts an electrophysiological recording of a probe detecting a dorsal grey matter.

FIG. 8 depicts an electrophysiological recording of a probe detecting a a dorsal root entry zone (DREZ).

### DETAILED DESCRIPTION OF THE INVENTION

The present disclosure provides compositions and methods for delivering therapies and therapeutic agents useful in the treatment of CNS defects, including injuries. As referred to herein, the terms “Central Nervous System” or “CNS” refer to the spinal cord and brain.

In one embodiment, the disclosure provides a method of identifying one or more delivery sites for a therapeutic agent in the CNS of a patient. Preoperative imaging data of the CNS of the patient is first obtained to determine boundaries of a defective region (e.g. injury site) in the CNS of the patient. One or more intraoperative ultrasound images of the region of injury are then obtained to delineate the boundaries of the injury site. The neuroelectrical activity in one or more injury tracts is then measured to identify zones of active neuron activity, inactivity, hypoactivity, and/or hyperactivity within an injury site or region of tissue. The ultrasound images are correlated with the measured neuroelectrical activity to identify one or more delivery zones for said therapeutic agent.

In another aspect of the disclosure, methods are provided to facilitate the cellular implant therapeutic agents within an injury site of a spinal cord. Several parameters are recorded and analyzed to identify a preferred environment within an injury site for delivery of the therapeutic agent. In preferred embodiments the environment is located within an acutely or chronically injured spinal cord, and the delivered therapy is the neuronal precursor cells or neurospheres of the invention for restoration or regeneration of tissue within the targeted site.

Preoperative Imaging

In various embodiments, injured sites of the CNS are identified by acquiring preoperative imaging data of the CNS. Various methods are known in the art to obtain preoperative imaging data. Preoperative imaging data includes Medical Resonance Imaging (MRI), computed tomography (“CT” or “CT scan”), x-ray data, and/or myelography.

MRI data are acquired by conventional procedures known in the art. In various embodiments, a strong magnetic field is applied to a patient's body. MRI is a non-invasive imaging method based on the use of radio waves and a magnetic field to provide cross-sectional images of the spinal cord. (see for example Falci et al., Journal of Spinal Cord Medicine, 22(3), 173-181 (1999)). The imaging information from the spinal cord is obtained and used to localize the site of the contusion or injury. In particular, the preoperative imaging data provides information regarding the size, shape and possible type of injury that the patient has suffered and facilitates the surgeons ability to localize the correct level of the spinal cord to expose. In addition, the preoperative imaging provides a view of the spinal cord's stability, the degree of spinal cord tethering, myelomalacic change, true cystic cavitation, and cerebrospinal fluid flow.

In some embodiments, the entire spinal cord can be imaged. In further embodiments, different slices of the spinal cord (e.g. the cervical, thoracic, or lumbar regions) can be imaged. Different depths of the spinal cord also can be imaged.

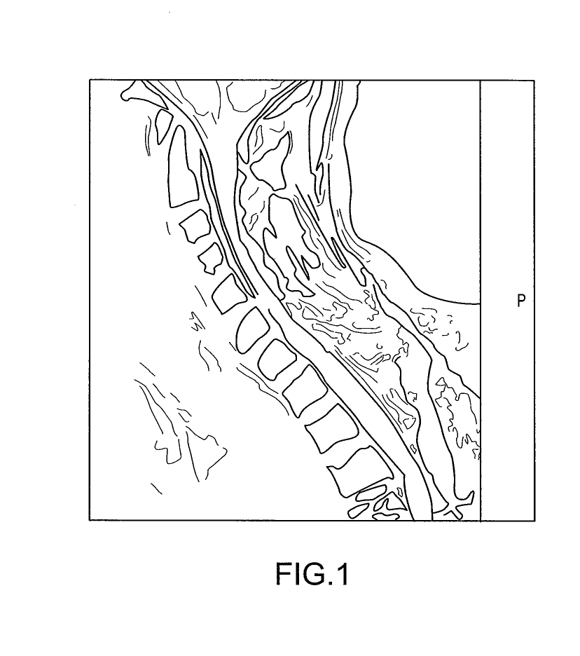

FIG. 1 depicts an exemplary MRI from a patient having a spinal cord injury. The MRI depicts three basic zones within an acutely injured spinal cord: a zone having reduced electrical activity, a zone having hyperactive electrical activity and a zone having normal electrical activity.

The zone having reduced electrical activity (electrophysiologic recording) is shown in the top panel of the recording data. The area of reduced activity corresponds to the cells within an injured spinal cord at a contusion site (reference letter A), i.e., the direct site where the injury occurred. The contusion site can be of various dimensions and is typically viewed on an ultra-sound as a dark space (corresponding to a signature of liquid and other similar density materials).

Intraoperative Ultrasound

Intraoperative ultrasound images of abnormal or injured regions of the CNS are acquired. In various embodiments, the preoperative images can then be correlated to information gathered via intraoperative ultrasound on the same spinal cord.

In various procedures, a laminectomy is performed on the injured spinal cord to expose the dura of the region of injury as determined from the imaging data. Intraoperative ultrasonography then provides a radiograph of the region of injury and provides a detailed view of possible nerve damage, myelomalacia, healthy or normal tissue. For example, intraoperative ultrasound can be used to access the size of the injury, regions of myelomalacia, location of spinal cord tethering and cord and rootlet motion. Cord tethering was recognized as a loss of normal subarachnoid space, adherence of the cord and rootlets to the dura, and the loss of the normal anterior-posterior motion of the spinal cord corresponding to the patient's heartbeat and CSF pulsations. Myelomalacia can be recognized as regions of hyperechoicity. The region and type of CNS (e.g. spinal locations) defects are thus precisely mapped out and identified.

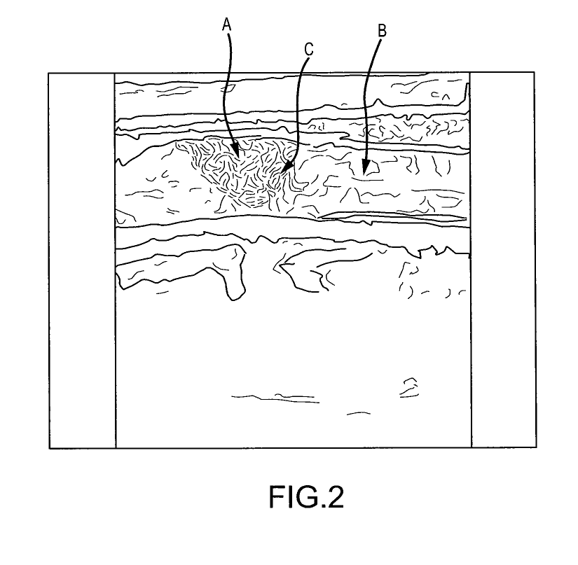

FIG. 2 depicts an exemplary intraoperative ultrasound of a spinal cord contusion. A contusion site can be seen in the ultra-sound as a dark space (corresponding to a signature of liquid and other similar density materials). Normal, uninjured tissue is defined by the lighter-colored space bordering the contusion area. The specific location and border region of injured tissue can thus be identified.

Measuring Neuroelectrical Activity

Using this information regarding the patient's injury site, the neuroelectrical activity of injured cells can then be determined within zones of the injury site to identify active neuron activity. Specific tracts of tissue can be detected. In particular, correlating imaging data and/or ultrasound data with the measured neuroelectrical activity of cells at the injury site is used to locate specific environments of cellular activity. Based on this correlation, a therapeutic delivery zone or zones within the injury site for delivery of therapeutic agents can be identified, thus increasing the prospect of a positive therapy result. The electrical signals can be used to identify sites for administration of additional therapies during surgery, without requiring estimation of the location and border of the injury site that would otherwise require a separately acquired ultrasound.

In additional embodiments, detection of neuroelectrical activity can be used to identify the location of treatment sites independently of additional imaging methods. For example, the neuroelectrical activity of the spinal cord can be detected at and surrounding a region of damaged tissue in the spinal cord to identify zones of active neuron activity or inactivity neuronal activity. In further aspects, hyperactivity and hypoactivity can also be detected. Therapeutic compounds can then be administered to the site identified by neuroelectrical activity.

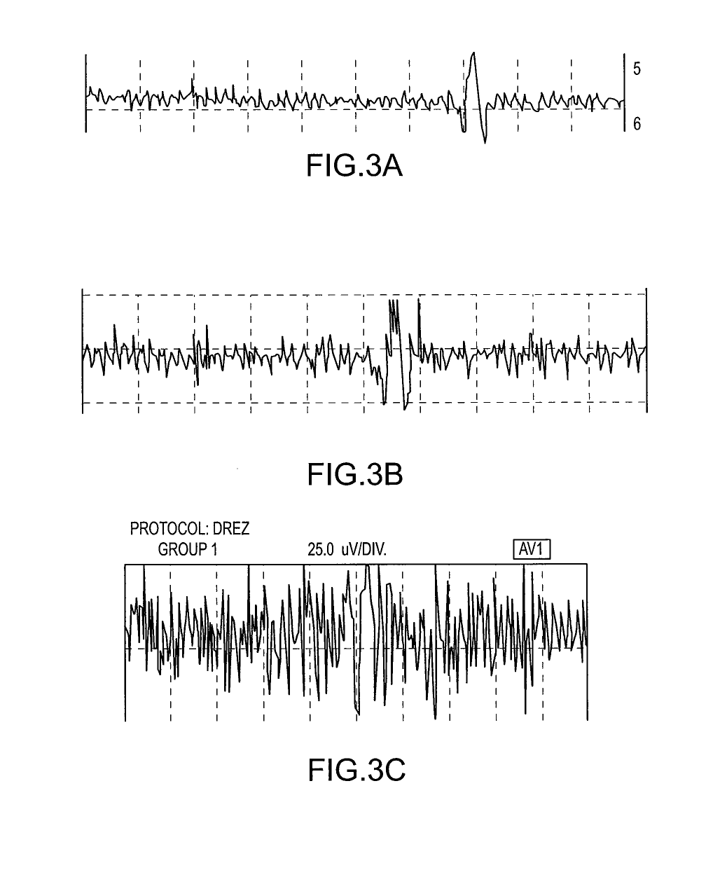

In one embodiment, zones at an acute injury site can be identified. As illustrated in FIG. 3, three basic zones within the acutely injured spinal cord were identified: a zone having reduced electrical activity, a zone having hyperactive electrical activity and a zone having normal electrical activity. The zone having reduced electrical activity (electrophysiologic recording) is shown in FIG. 3A. The area of reduced activity corresponds to the cells within an injured spinal cord at a contusion site depicted in FIG. 3D at position A, i.e., directly at the contusion site. Contusion sites can be of various dimensions and are typically viewed on an ultra-sound as a dark space (corresponding to a signature of liquid and other similar density materials).

An area of cells showing normal or healthy neuroelectrical activity is typically caudal to a contusion site. A typical electrophysiologic recording of spontaneous neuroelectrical activity for normal or healthy cells from within the normal zone is shown in FIG. 3B. This region corresponds to healthy cells in the light-colored region of FIG. 3D (noted by reference letter B).

A third zone of cells adjacent to, or bordering, the acute injury site shows a third type of electrical activity. This bordering zone of cells around the contusion site show hyperactive neuroelectrical activity, as is shown in FIG. 3C. The area of hyperactivity corresponds to the cells within the area of the ultrasound labeled with reference letter C of FIG. 3D.

The third zone of hyperactive cells in acute injuries one year or less, more typically six months or less, and most typically in three months or less, is reduced to less than normal organized electrical activity over time. This pattern suggests a substantial loss of neurons within this bordering zone, until in chronic injury sites, no hyperactive neuroelectrical activity is shown or is anticipated. The border zone of cells represents a region which progressively undergoes cell death (apoptosis and necrosis) over a period of time (typically less than one year). As such, this region of cells represents a source of tissue at an acute injury location that is undergoing progressive neuronal deterioration, and can be a favorable region for administering therapeutic agents.

In various embodiments, the border region of an acute injury site represents the zone of cells most likely to enhance the effects of the injury, as these cells are shown to be the most likely to progressively die over time (within one year of injury). As such, this border zone of cells represents a target site within the acutely injured cord for slowing cell death. Therapies intended to minimize cell death and loss can be delivered directly to the border zone of the acutely injured cord, thereby concentrating the effectiveness of regenerative and/or non-apoptotic drug approaches.

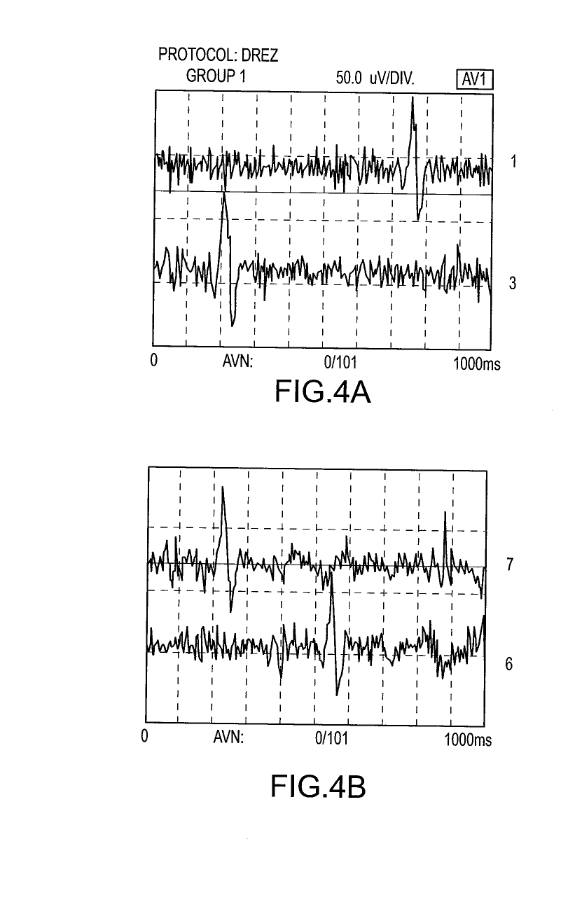

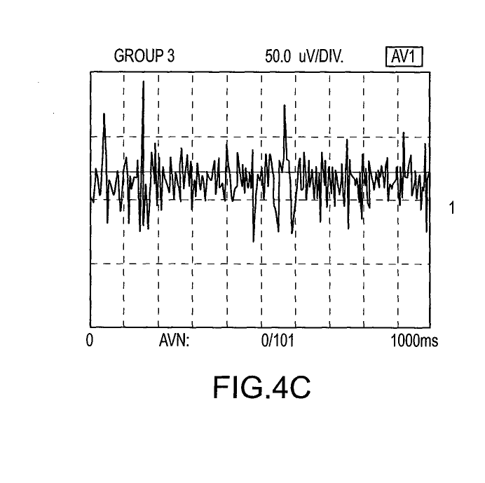

Different types of healthy tissues can be identified. For example, specific injuries within different types of neuronal tissue can also be detected from electrophysiologic recordings of specific types and tracts of tissues. FIG. 4A shows an electrophysiologic recording of uninjured tissue in the spinal thalamic tract. FIGS. 4B and 4C, respectively, depict electrophysiologic recordings of uninjured tissue in the posterior column lateral and medial, respectively. The posterior column-medial lemniscus pathway is the sensory pathway responsible for transmitting fine touch and conscious proprioceptive information from the body to the cerebral cortex.

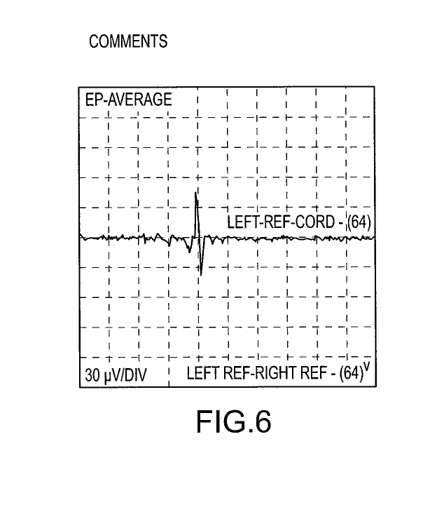

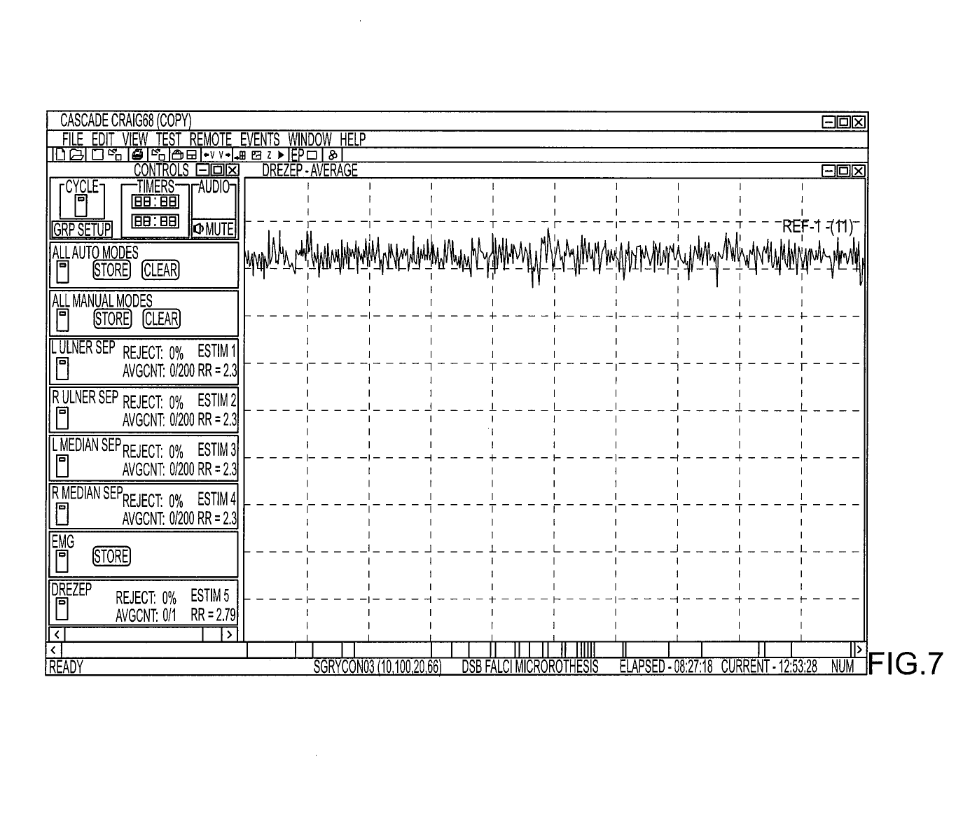

Changes in the electrical signals measured in specific tracts or columns in a patient allow the specific location of an abnormality or injury to be identified with high precision. FIG. 6 depicts an example of extreme myelomalacia and cystic necrosis in the tissue of a patient. The abnormal tissue is identified by the absence of organized neuronal electrical activity of low energy. Therapeutic agents can thus be targeted directly to a specific injury site either with or without ultrasound imaging, and a specific tract within the injury zone identified by neuroelectrical measurements.

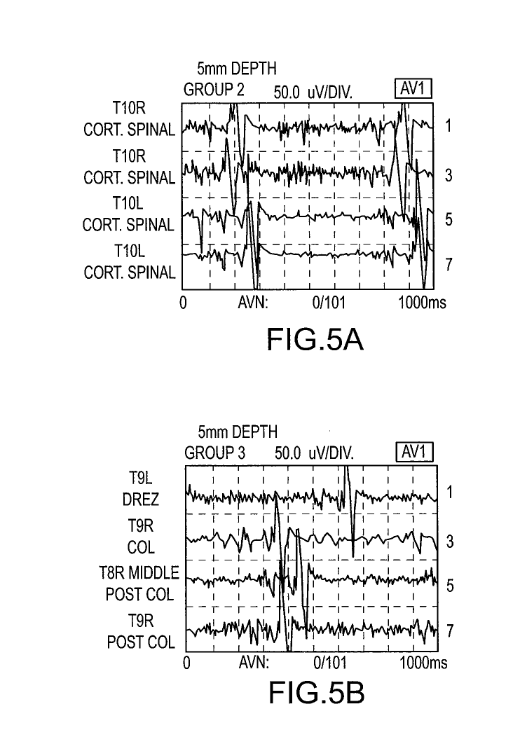

Alternatively, different regions of spinal cord tissue can be distinguished by measurement of neurophysiological signals. For example, cellular tracts can be distinguished from dorsal root entry zones (DREZ). FIG. 5A depicts an electrophysiologic recording of the corticospinal tract of a patient, and FIG. 5B depicts an electrophysiologic recording of the dorsal grey matter of a patient. Each of FIGS. 5A and 5B show a high frequency signal corresponding to the firing of multiple nerve cells. FIG. 5B however also includes an underlying slow frequency signal corresponding to organized cellular firing represented in the tract. The high frequency nerve signal combined with the low frequency cellular signal identifies the tissue as a tract. Similarly, FIG. 7 also depicts a high frequency signal corresponding to the fast firing of multiple nerve cells overlapping a slow frequency signal corresponding to organized cellular activity. The high frequency nerve signal combined with the low frequency cellular signal identifies the tissue as a tract in the column.

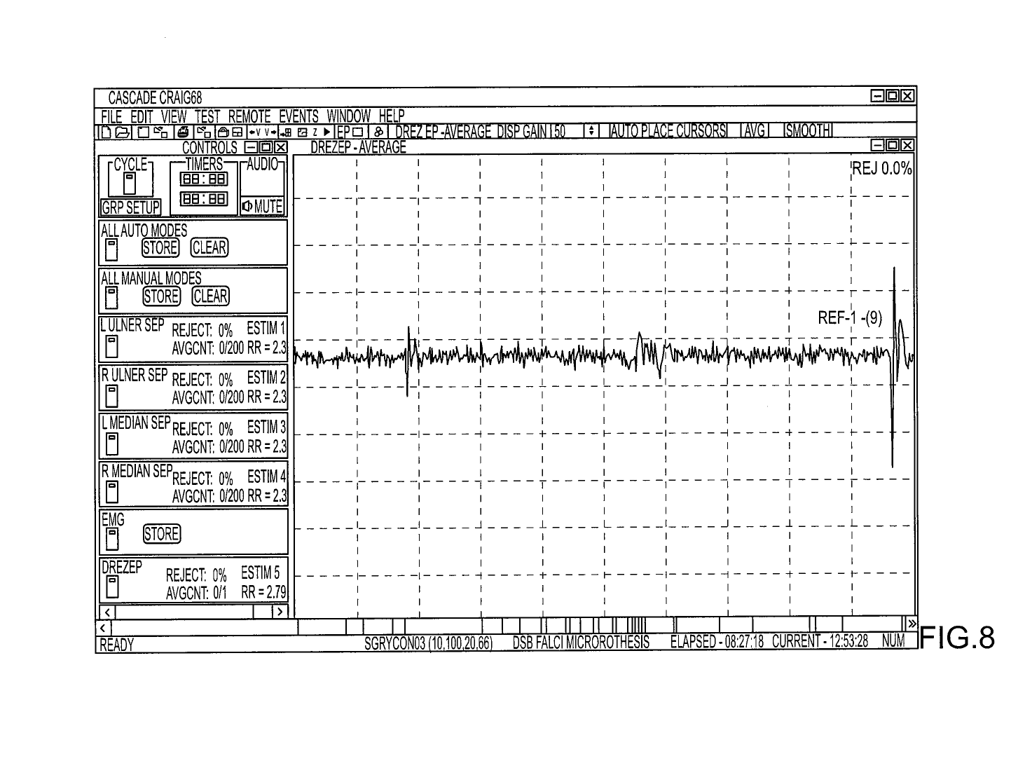

By contrast, FIG. 8 depicts the signal obtained from grey matter of a dorsal root entry zone (DREZ). The high frequency corresponds to fast firing of nerves at the DREZ. However, there is not underlying slow frequency overlapping the high frequency signal. The DREZ signal is thus readily distinguished from cellular tracts.

Therapeutic Agents

Therapeutic agents can be selected and targeted to specific types of injured tissue based on the correlation of imaging methods and neuroelectrical measurements.

Injury to the spinal cord often results in mechanical killing and damage to axons. Damage can include both grey and/or white matter within the spinal column. Typically, injuries to the cord include a second wave of damage, often caused by inflammation within the initially damaged area. The end result is a state of disrepair within the cord. Often axons are not functional due to disconnection or loss of insulating myelin. In other circumstances, glial scars form within the area of damage. In other cases, axons remain intact and myelinated, but carry insufficient signal volume to convey useful directives to the brain or muscles.

Recently, it has been determined that as little as 10% of the standard axon complement would provide the capacity for an injured patient to walk, or that limiting an injury site to 0.5 inches×0.5 inches could have a significant impact on a patient's quality of life (McDonald, Repairing the Damaged Spinal Cord, 281(3):64-73). As such, administration of therapies of the spinal cord at targeted locations provide enhanced treatment of spinal cord injuries.

Various therapeutic agents can be delivered to a specific CNS location using the methods disclosed herein. For example, axon growth stimulating factors, inhibitor-neutralizing antibody or equivalent small molecules, netrins, corticosteroids, nerve grafts, extracellular matrix components, scaffolding components (e.g. Schwann cells), genetically engineered fibroblasts, and various progenitor cells, for example mesenchymal stem cells, embryonic stem cells, etc. In various embodiments, combinations of biologic factors and cell populations will be delivered together to maximize the targeted environment and provide building blocks for repair within the injury site. Examples of these therapies include, but are not limited to, those described in Tator et al., Neurosurgery 59(5) November 2006. These include neuropharmaceuticals as well as cellular therapies.

Additional therapies can include embryonic stem cells, fetal olfactory bulbs, autologous olfactory mucosa, and stem cells described in Tator et al. (supra) can be used. Pharmaceuticals administered can include minocycline, cethrin, and ATI355.

In various embodiments, stem cells can be administered. For example, bone marrow stem cells can be administered as whole stem cells. Bone marrow cells can contain a mixture of hematopoietic cells, various mononuclear cells, such as macrophages, and marrow stromal cells. In other embodiments, peripheral blood stem cells can be administered. In further embodiments, human stem cells, including neuronal stem cells or pluripotent cells, human embryonic stem cells, or fetal Porcine Stem Cell Xenotransplants can be administered. Additional cellular therapies can include human fetal spinal cord cells, olfactory ensheathing glia or olfactory Bulb cells can be administered.

In still other embodiments, a rho antagonist can be administered. Exemplary rho antagonists include kinase inhibitors (e.g., cethrin). Alternatively, an anti-Nogo-A inhibitor can be administered to the Nogo inhibitory protein associated with myelin. Nogo inhibitors can include antibody or antibody-fragment based inhibitors.

In certain examples, cellular therapies can include isolation and expansion of neural precursor cells in a manner that maintains and optimizes their capacity to restore and/or regenerate function within an injured CNS, and in particular within an injured spinal cord. Embodiments disclosed herein illustrate the utility of targeting these cells into various types of CNS injury, and in particular, spinal cord injury.

In certain embodiments, therapeutic agents can be administered to regions of damaged tissue to prevent expansion of initial damage and promote secondary damage. For example, therapeutic agents that block excitoxic injury can be administered directly to the injured cells or border region of cells. The administration of a therapy can be administered, for example, to the hyperactive neuroelectrical signal observed at the border region of an injury in FIG. 3C.

Alternatively, axonal regenerative therapy can be provided to the patient. For example, therapeutic agents that overcome natural inhibitors of regeneration can be provided to the axon at a specific location identified herein. Agents that induce axonal growth can also be administered. For example, peripheral nerve bridges and neurotrophic factors can be added to a cell.

In additional embodiments, compounds can be implanted into dead tissue cysts that can serve as scaffolds for axon regeneration.

In other embodiments, treatment may be delivered to demyelinated tissue. For example, chemicals can be delivered that prevent nerve impulses from dissipating at demyelinated areas. Further, agents can be provided that spur surviving oligodendrocytes to remyelinate axons. For example, exogenous myelin-forming cells can be transplanted.

In further embodiments, undiffentiated stem cells or precursor cells can be administered directly to an injury location identified by the techniques described herein. For example, neurospheres can be administered to restore or regenerate damaged CNS tissue. In general, these cells are shown to have potent regenerative capacity in the treatment of various CNS injuries. Embodiments of the invention provide methods for procurement of the precursor cells to the neurospheres. Target tissue having neuronal precursor cells is harvested and dissected in accordance with approved techniques. For example, embryonic/fetal tissue is identified and dissected under sterile conditions. The gestational age of the tissue, including size and anatomical landmarks, is used to identify areas of expected neuronal precursor cells. The procedure can be accomplished under a dissection microscope. In general, pieces of the forebrain and spinal cord are dissected from the embryonic/fetal tissue, freed of meninges and visible blood vessels, and rinsed in serum free culture medium. Note that preferred embodiments utilize human embryonic/fetal tissue sources.

Various combinations of therapies are discussed in McDonald, supra, which is herein incorporated by reference in its entirety.

Screening Therapeutic Agents

In various embodiments, injured cells can be screened to determine the effectiveness of a given therapeutic agent on a specific tract of cells.

In one example, effective treatments can be screened or tested to identify specific treatment targets for cells at the injury site, border zone, and normal zone depicted in FIGS. 3A-D. A sample of both the hyperactive, neuronal degenerating cells in the border zone and normal cells in the normal zone can be taken from a patient. The sample can be taken by any method known in the art. In certain embodiments, samples can be taken by biopsy from the patient, for example a 1 mm×1 mm×2 mm section of target tissue in the target zone. Note also that each sample is isolated and treated separately—to minimize any potential for cross-contamination between samples. The samples removed from the patient are analyzed using one or more biochemical/molecular techniques to identify effectors present or at higher concentrations in the border zone than in the normal zone sample. These techniques, for example differential gene expression, protein separation by electrophoresis, etc., can be found in any of several well-known references, such as: Molecular Cloning: A Laboratory Manual (Sambrook et al. (1989)); Gene Expression Technology (Methods in Enzymology, Vol. 185, edited by Goeddel (1991) Academic Press, San Diego, Calif.); “Guide to Protein Purification” in Methods in Enzymology (Deutshcer, 3d., (1990) Academic Press, Inc.), PCR Protocols: A Guide to Methods and Applications (Innis et al. (1990) Academic Press, San Diego, Calif.); and Gene Transfer and Expression Protocols, pp 109-128, ed. Murray, The Humana Press Inc., Clifton, N.J.). Note also that the analysis may alternatively provide data that the hyperactive border zone cells are devoid of neuronal degenerating effectors or express a potential inhibitor of neuronal degeneration at a lower concentration than in the normal tissue. In any event, the methods will provide the identification of neuronal degeneration.

In alternative embodiments, injured and normal specific spinal tracts can be screened for targets or cellular markers. For example, samples of injured and normal white matter or dorsal grey matter can be biopsied and tested against various treatments prior to providing the treatment to the patient. Alternatively, injured or normal tissue of different tracts can be tested to identify markers or effectors specific to an injury location. Samples removed from the patient can be analyzed using one or more biochemical/molecular techniques to identify effectors present or at higher concentrations in various tracts and injury zones.

In alternative embodiments, a therapeutic agent can be screened for effectiveness on different spinal tracts. For example, samples of injured and normal white matter or dorsal grey matter can be biopsied and tested against various treatments prior to providing the treatment to the patient. The effectiveness of a given therapy on a specific cell type can be determined by in vitro assays.

The disclosure will be more readily understood by reference to the following example, which is provided by way of illustration and is not intended as limiting.

### EXAMPLE

MRI, Ultrasound and Electrical Signature Map Target Sites For Delivery of A Cellular Therapy in an Injured Spinal Cord

### Clinical Materials and Methods:

A combination of data from one or more patients has been used to illustrate one embodiment of the present invention. Data was selected to illustrate one or more aspects for targeting and delivery of a cellular or biologic therapy to an injury site. Data for the present invention was obtained from patients who have sustained traumatic thoracolumbar spinal cord injuries.

Patients eligible for cellular or biological therapy to the spinal cord typically have had an injury causing significant damage to one or more regions of the spinal cord.

### Preoperative Procedures:

A patient scheduled to undergo the methods of the present invention will undergo preoperative evaluation with one or more of: plain x-ray, magnetic resonance imaging (MRI), and/or cat scan (CT) myelography. The combination provides an evaluation of the spinal cord injury site. Prior to surgical treatment in accordance with the present invention, a patient would undergo pharmacologic treatment appropriate for spinal cord injury such as methylprednisolone in the acute phase, and any other medications needed to treat spasticity, autonomic dysreflexia, neuropathic pain, or any other symptoms related to the spinal cord injury including administration of oral tricyclic antidepressants (TCA), antiseizure medication, Baclofen, Klonopin, and narcotic analgesics. In some instances, a pump can be placed for intrathacal infusion of narcotics, Baclofen, Clonidine or local anesthetic. FIG. 1 illustrates an MRI from a patient having an injury to the spinal cord consistent with a contusion and regions of cystic necrosis, myelomalacia and regions of healthy cord. The MRI gives an initial indication as to the dimensions of the injury site, time frame from which the injury occurred, potential boundaries within which a cellular or biologic therapy would be delivered.

Intraoperative Ultrasound and DREZ Recording

Multilevel laminectomies were performed on the patient as is known to one of skill in the art. Briefly, laminectomies were performed to expose the spinal cord at the level of injury, as well at uninjured levels cephalad and caudal. Spinal levels for potential delivery of a cellular or biologic therapy were determined by the preoperative procedures described above. An intraoperative ultrasonography was performed to better identify the injury site.

Measuring Neuroelectrical Activity

The neuroelectrical activity was measured to determine the electrical signal at different locations of the acute injury. An active electrode was inserted into different locations of the DREZ tissue. The active electrode used was a 25 mm TECA MF 25 monopolar electrode with the distal 2 mm exposed. The electrode was implanted at each tissue location with use of the intraoperative microscope to a 2 mm depth. Ground and reference Glass subdermal (EEG) electrodes were placed in exposed paraspinous muscle bilaterally. Spontaneous electrophysiologic recordings were made with a Cadwell Spectrum 32 evoked potential averager at a gain setting of 50 with the high frequency filter set at 3 KHz and the low frequency filter at 100 Hz. The recordings were one second in duration. Additional evaluation with fast fourier transform, root mean square analysis, and spindle analysis were made.

1. A method of delivering a therapeutic agent to the central nervous system (CNS) of a patient comprising:
obtaining preoperative imaging data of the CNS of the patient to determine boundaries of a region of damaged tissue in said the CNS of the patient;
obtaining one or more intraoperative images of said region of damaged tissue;
measuring neuroelectrical activity in and surrounding the region of damaged tissue in one or more tracts of grey and white matter to identify zones of active neuron activity, inactivity, hypoactivity, and/or hyperactivity;
correlating said ultrasound images with the measured neuroelectrical activity at and surrounding the region of damaged tissue to identify one or more delivery zones for said therapeutic agent;
delivering the therapeutic agent to the one or more delivery zones.

2. A method of delivering a therapeutic agent to the central nervous system (CNS) of a patient comprising:
measuring neuroelectrical activity in and surrounding the region of damaged tissue in the spinal cord of said patient to identify zones of active neuron activity, inactivity, hypoactivity, and/or hyperactivity to identify one or more delivery zones for said therapeutic agent;
delivering the therapeutic agent to the one or more delivery zones.

3. The method of claim 1, wherein the preoperative image data is selected from the group consisting of a MRI, a CT scan, and an X-ray scan.

4. The method of claim 1, wherein the preoperative image data is a MRI.

5. The method of claim 1, wherein said one or more tracts are selected from the group consisting of the spinal thalamic tract, the posterior column, the corticospinal tract, and dorsal grey matter.

6. The method of claim 1 or 2, wherein said delivery zone comprises a regions of hyperactivity.

7. The method of claim 1 or 2 wherein the therapeutic agent promotes axonal regeneration.

8. The method of claim 1 or 2, wherein the therapeutic agent promotes remyelination.

9. The method of claim 1 or 2, wherein the therapeutic agent inhibits nerve impulses from dissipating in demyelinated areas.

10. The method of claim 1 or 2, wherein the therapeutic agent inhibits exotoxic shock.

11. The method of claim 1 or 2, wherein the therapeutic agent comprises stem cells.
